(19) United States
(12) Patent Application Publication
(10) Pub. No.: US 2005/0276676 A1
(43) Pub. Date: Dec. 15, 2005

(54) Orthodpedic or dental device

(76) Inventors: Ofer Mardinger, Savion (IL); Gabriel Chaushu, Rishon Letzion (IL)

Correspondence Address: BROWDY AND NEIMARK, P.L.L.C.;624 NINTH STREET, NW, SUITE 300, WASHINGTON, DC, 20001-5303, US

(21) Appl. No.: 10/867,206

(22) Filed: Jun. 15, 2004

(51) Int. Cl. A61B 17/86; F16B 35/04

(52) U.S. Cl. 411/411; 606/73; 433/174

(57) ABSTRACT

A medical device having an outer surface, at least a portion of the outer surface being provided with a continuous thread. The thread has a feature that is not constant in the portion of the outer surface. The feature that is not constant may be, for example, the spacing between turns of the thread, the height of the thread, the thickness of the thread, or the pitch of the thread. The feature that varies may vary continuously or discontinuously. The device may be, for example, a screw, a dental implant, an orthopedic implant, a maxillo-facial device, an orthodontic device, a maxillo-facial fixation screw, a nailing device, or a sinous lift device. Varying a feature of the thread allows the device to be adapted for insertion in to bone having varying properties along the length of the device.

### FIELD OF THE INVENTION

The present invention relates to dental and orthopedic devices, and more specifically to such devices having a threaded outer surface for screwing into bone tissue.

### BACKGROUND OF THE INVENTION

Dental and orthopedic devices typically have a cylindrical main body that is provided with a helical screw thread on its outer surface in order to allow the device to be screwed into a pre-bored hole in bone tissue.

Bone tissue often degenerates around the outer surface of an implanted device, a phenomenon known as “pressure resorption”. In extreme cases, pressure resorption leads to complete detachment of the device from the surrounding bone tissue. Pressure resorption is believed to occur at those regions on the surface of the device in which the pressure exerted on the adjacent bone tissue by the device is too low or too high. In general, a relatively low pressure is required in compact bone tissue in order to avoid pressure resorption, while a relatively high pressure is required in cancellous bone. The pressure exerted on the surrounding tissue is determined, inter alia, by the diameter of the device relative to the diameter of the pre-bored hole, as well as by the height, spacing and pitch of the thread.

U.S. Pat. No. 5,588,838 to Hansson et al. discloses a device having a cylindrical main body and a conically flaring upper portion. The outer surface of the main body and the neck are both provided with screw threads. The presence of the thread on the neck decreases the tendency of pressure resorption around the neck. The thread has uniform pitch, height and spacing along the surface of the main body. Similarly the thread in the neck has uniform pitch, height and spacing. The features of the thread on the neck surface maybe different from those along the main body surface. The thread along the main body surface is not continuous with the thread along the neck surface.

### SUMMARY OF THE INVENTION

The present invention provides a device for insertion into bone tissue. The outer surface of the device is provided with an essentially helical screw thread. The thread may extend continuously along almost the entire length of the outer surface, or be restricted to one or more segments along the length of the device, the thread being continuous in each of the one or more segments. In accordance with the invention, at least one feature of the thread is not constant along the entire length of a continuous segment of the thread. For example, the spacing between adjacent turns of the thread may vary along a continuous segment of the thread. As another example, the height or thickness of the thread may vary along a continuous segment of the thread. As yet another example, the pitch of the thread turns may vary along a continuous segment of the thread. The varying feature may vary continuously along the length of the thread, or there may be an abrupt change in the feature at one or more points along the continuous thread.

Varying a feature of the thread along its length allows the thread to be optimally matched to varying features of the surrounding bone tissue into which the device is to be inserted. In particular, it allows the pressure exerted by the device on the surrounding tissue to be optimally selected for avoiding pressure resorption. For example, a segment of the thread having closely spaced turns generates a larger pressure than a segment of the thread in which the turns are widely spaced. Thus, a segment of the thread having closely spaced turns is more suitable for insertion into cancellous bone tissue, than a segment of the thread having widely spaced turns. Similarly, a segment of the thread having widely spaced turns is more suitable for compact bone tissue.

The device may be, for examples a screw, a dental implant, an orthopedic implant a maxillo-facial device, an orthodontic device, a maxillo-facial fixation screw, a nailing device, or a sinous lift device. The device of the invention may be used in any orthodontic or dental application where a device immobilized in bone is required. For example, the device may be immobilized in a jaw bone for the attachment of a dental prosthesis. It may also be used as a screw for the attachment of splint plates to bone tissue for setting a fractured bone. It may also be used in orthodontic procedures, for example, for immobilizing an orthodontic device. It may also be used for immobilizing the mandible.

The invention thus provides a medical device having an outer surface, at least a portion of the outer surface being provided with a continuous screw read having a feature that is not constant in the portion of the outer surface.

### BRIEF DESCRIPTION OF THE DRAWINGS

In order to understand the invention and to see how it may be carried out in practice, a preferred embodiment will now be described, by way of non-limiting example only, with reference to the accompanying drawings, in which:

FIG. 1 shows a device in accordance with one embodiment of the invention.

### DETAILED DESCRIPTION OF THE INVENTION

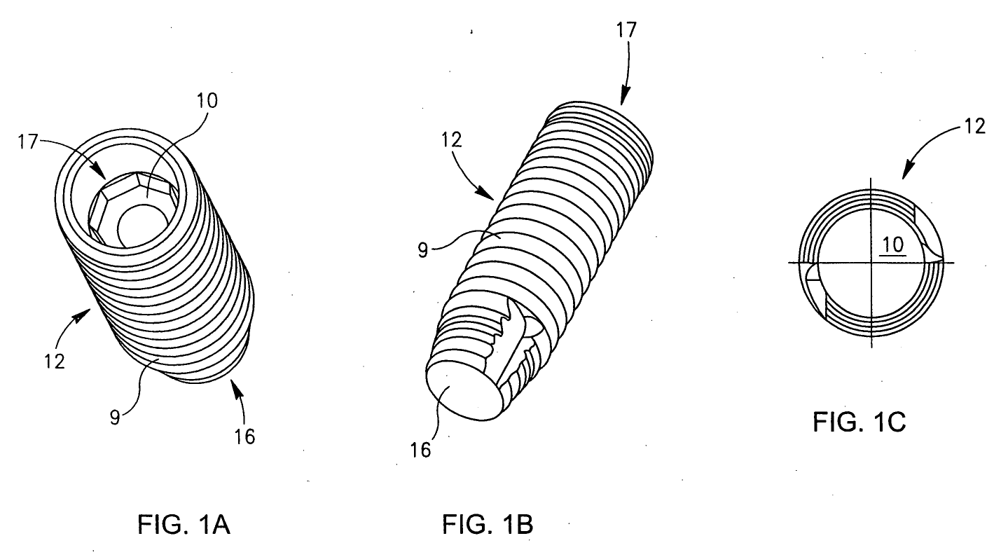

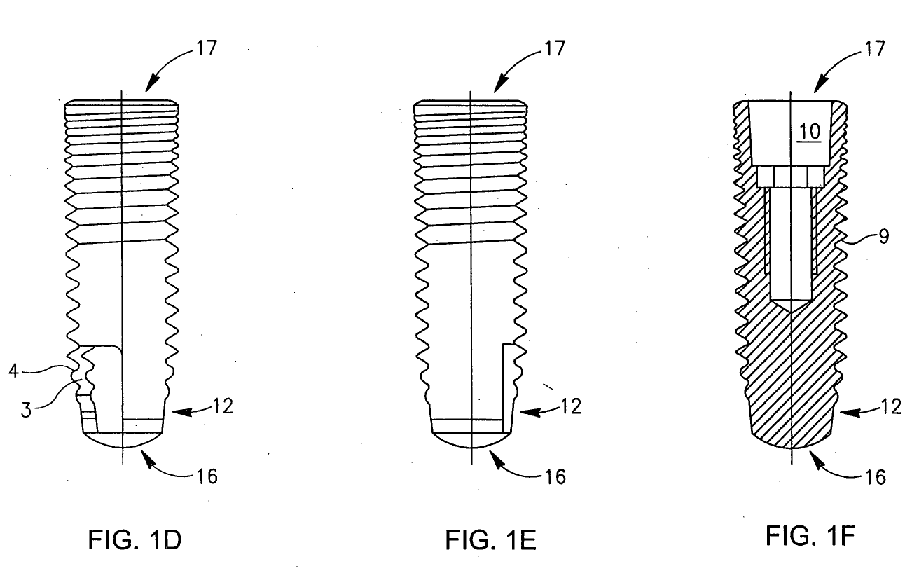

FIG. 1 shows an orthopedic or dental device 12 in accordance with one embodiment of the invention. The device 12 is shown in a perspective view from above in FIG. 1a, a perspective view from below in FIG. 1b, and in a top view in FIG. 1c. The device 12 is shown in side views in FIGS. 1d and e, and in longitudinal section in FIG. 1f. The device 12 has a first end 16 and a second end 17. The device 12 has a generally cylindrical shape suitable for insertion into a pre-bored hole in a bone tissue. A longitudinal blind bore 10 inside the device 12 extends into the device from an opening in the second end 17. The bore 10 has a shape that is configured to accommodate an attachment part of an abutment (not shown) designed to bridge the soft tissue covering the bone tissue and to form an attachment site of a prosthetic device, as is known in the art.

As can be seen in FIG. 1, the outer surface of the device 12 is provided with an essentially helical screw thread 9. In the embodiment of FIG. 1, the thread extends along almost the entire length of the outer surface, and is continuous along the entire threaded region of the outer surface. In accordance with the invention, at least one feature of the screw thread is not constant along the entire length of the continuous thread. In FIG. 1, the spacing between adjacent turns of the thread 9, as well as the height of the thread 9 and the thickness of the thread 9 varies along the length of the thread. The spacing between adjacent turns of the thread 9 decreases continuously as the thread progresses from the first end 16 towards the second end 17, from about 0.7 mm near the first end 16 to about 0.25 mm near the second end 17. The width of the threads as well as the height of the threads also decreases as the thread progresses from the first end to the second end 17. This is by way of example only and, in accordance of the invention, any feature of the thread may be non uniform, along the length of the thread. As yet another example, the pitch of the thread turns may vary between 45° and 60°. The varying feature may vary continuously along the length of the thread, or there may be an abrupt change in the feature at one or more points along the continuous thread

The device 12 may have at its first end 16 cutting edges 4 together with chip-collecting cavities 3. The cutting edges 4 and the chip-collecting cavities 3 allow the device 12 to function as a self-tapping screw for cutting a new thread in the bone or adjusting an already cut thread in the tissue, as is known in the art.

Varying a feature of the thread 9 along its length allows the thread to be optimally matched to varying properties of the surrounding bone tissue into which the device is to be inserted. In particular, it allows the pressure exerted by the device on the surrounding tissue to be optimally selected for avoiding pressure resorption. For example, a segment of the thread having closely spaced turns generates a larger pressure than a segment of the thread in which the are widely space. Thus, a segment of the thread having widely closely spaced turns is more suitable for insertion into cancellous bone tissue, than a segment of the thread having widely spaced turns. Similarly, a segment of the thread having widely spaced turns is more suitable for compact bone tissue. Thus, the region of the device 12 near the first end 16 where the turns of the thread are widely spaced is preferentially adapted for insertion into cancellous bone tissue rather than compact bone tissue. Similarly, the region of the device 12 near the second end 17, in which the turns of the thread 9 are narrowly spaced is preferentially adapted for insertion into compact bone tissue rather than cancellous bone tissue. Thus the device 12 is suited for insertion into a bone tissue such that after insertion, the region of the device 12 near the first end 16 is surrounded by cancellous bone, while ite region near the second end 17 is surrounded by compact bone.

1. A medical device having an outer surface, at least a portion of the outer surface being provided with a continuous screw thread having a feature that is not constant in the portion of the outer surface.

2. The device according to claim 1 wherein the feature that is not constant is selected from the group comprising:
(a) a spacing between turns of the thread;
(b) a height of the thread;
(c) a thickness of the thread; and
(d) a pitch of the thread.

3. The device according to claim 1, the device being selected from the group comprising:
(a) a screw;
(b) a dental implant;
(c) an orthopedic is plant;
(d) a maxillo-facial device;
(e) an orthodontic device;
(f) a maxillo-facial fixation screw;
(g) a nailing device; and
(h) a sinous lift device.

4. The device according to claim 1 wherein the feature varies continuously in the portion of the outer surface.
